(19) United States
(12) Patent Application Publication
(10) Pub. No.: US 2016/0178884 A1
(43) Pub. Date: Jun. 23, 2016

(54) LENSES UNIT FOR ENDOSCOPE, AND ENDOSCOPE EQUIPPED WITH THE SAME

(71) Applicant: Panasonic Intellectual Property Management Co., Ltd., Osaka (JP)

(72) Inventors: Yasuyuki Hanada, Fukuoka (JP); Saturo Miyanishi, Fukuoka (JP); Akinori Osada, Fukuoka (JP); Yoshio Matsumura, Osaka (JP)

(21) Appl. No.: 14/927,152

(22) Filed: Oct. 29, 2015

(30) Foreign Application Priority Data
Dec. 22, 2014 (JP) 2014-259324

(51) Int. Cl. G02B 23/24; A61B 1/00; A61B 1/055; G02B 7/02; G02B 27/00

(52) U.S. Cl. CPC G02B 23/243; G02B 7/021; G02B 7/025; G02B 27/0025; A61B 1/055; A61B 1/00121; A61B 1/00188

(57) ABSTRACT

A lenses unit for an endoscope includes a lens barrel; a lens serving as a front group lens and lenses serving as a rear group lens, which are housed inside the lens barrel; and an aperture arranged between the front group lens and the rear group lens. An imaging-side final surface of the rear group lens has a structure in which the imaging-side final surface is fixed to a cover glass of an image pickup device with an adhesive layer. A focal distance f_{F} of the front group lens, a focal distance f_{B} of the rear group lens, a focal distance fₑₗ of an entire optical lenses group, a total optical length OL of the optical lenses group, and a metal back MB of the optical lenses group satisfy a relationship of fₑₗ/f_{F}<0, fₑₗ/f_{B}>0, and OL/MB>7.0.

### BACKGROUND OF THE INVENTION

1. Field of the Invention

The present invention relates to a small-diameter lenses unit for an endoscope used for, for example, the medical field, and an endoscope equipped with the same.

2. Description of the Related Art

In the related art, in the medical field or the industrial field, endoscopes for imaging the interior of a human being's body or the inside of a device or a structure have spread. As these types of endoscopes, there are electronic endoscopes in which an imaging unit including an imaging lens and an image pickup device is mounted on an insert section inserted into the inside of an object to be observed. In the electronic endoscopes, the light from a region to be imaged is image-formed on an imaging surface of the image pickup device by the imaging lens, and the image-formed light is converted into an electrical signal by the image pickup device, and is transmitted to an external image processing device or the like as a video signal via a signal cable. In the endoscopes, for expansion in the range of an object to be observed, reduction in the burden of a person to be observed, the simplification of structure, cost reduction, or the like, downsizing of the inset section has been mentioned as a challenge until now. In the electronic endoscopes, further downsizing and further diameter reduction of a distal end portion including the imaging unit have become important.

In order to achieve downsizing of optical instruments, such as an endoscope, for example, Japanese Patent Unexamined Publication No. 2013-200537 discloses an imaging mechanism and an endoscope in which a plano-concave lens is provided on an image pickup device side of a lenses unit, and a planar portion of the plano-concave lens is made to project from an end of the lens barrel and is fixed to a cover member that covers the surface of an image pickup device. Japanese Patent No. 3426378 discloses an endoscope objective lens having three lens groups capable of reducing distortion even in a spherical lens only and realizing cost reduction.

The imaging unit of the electronic endoscopes is generally configured to perform focus adjustment between the imaging lens and the imaging surface of the image pickup device, using a lens barrel that houses the imaging lens, and a frame body (distal end hard part) that holds the image pickup device. If realization of excellent optical performance is intended, in the configuration of the related-art imaging lens, a back focus becomes long. As a result, it is necessary to secure a predetermined distance from a final surface of the imaging lens to a cover member surface of the image pickup device. For this reason, it is difficult to realize the structure of fixing the imaging lens and the image pickup device with an adhesive or the like for the purpose of downsizing. In the endoscope objective lens described in Japanese Patent No. 3426378, the total optical length is about 6.51 mm to 7.22 mm, the back focus is about 0.70 mm to 0.87 mm, and the back focus is longer than the total optical length. Therefore, the imaging lens and the image pickup device cannot be fixed without interposing a frame body or the like. In the imaging mechanism described in Japanese Patent Unexamined Publication No. 2013-200537, the plane of the final surface on the image pickup device side is configured to be fixed to the cover member of the image pickup device, but there is no refractive power in the final surface. Therefore, this cannot contribute to the convergence of rays from a photographic subject, and a reduction in the aberration of an optical system. For this reason, it is difficult to obtain desired optical performance while achieving downsizing of the imaging lens.

### SUMMARY OF THE INVENTION

An object of the invention is to provide a lenses unit for an endoscope and an endoscope that are able to realize the structure of fixing an imaging lens and an image pickup device with an adhesive or the like.

According to an aspect of the invention, there is provided a lenses unit used for an endoscope, including a lens barrel; a front group lens and a rear group lens housed inside the lens barrel; and an aperture arranged between the front group lens and the rear group lens. An imaging-side final surface of the rear group lens has a structure in which the imaging-side final surface is fixed to a cover glass of an image pickup device with an adhesive layer. A focal distance f_{F} of the front group lens, a focal distance f_{B} of the rear group lens, a focal distance fₑₗ of an entire optical lenses group including the front group lens and the rear group lens, a total optical length OL of the optical lenses group, and a metal back MB of the optical lenses group satisfy a relationship of fₑₗ/f_{F}<0, fₑₗ/f_{B}>0, and OL/MB>7.0.

According to another aspect of the invention, there is provided an endoscope including the above lenses unit for an endoscope; an image pickup device in which an imaging surface is covered with cover glass; an imaging-side final surface of the rear group lens in the lenses unit for an endoscope; and an adhesive layer formed by an adhesive resin with which the cover glass of the image pickup device is fixed.

According to the invention, the structure of fixing the imaging lens and the image pickup device with an adhesive or the like in the imaging unit of the endoscope can be realized.

### BRIEF DESCRIPTION OF DRAWINGS

FIG. 1 is an overall configuration view of an endoscope system using an endoscope related to an embodiment of the invention;

FIG. 2 is a perspective view illustrating the configuration of a distal end portion of the endoscope related to the present embodiment;

FIG. 3 is a sectional view of the distal end portion of the endoscope related to the present embodiment;

FIG. 4 is a perspective view illustrating the configuration of portions excluding mold resin in the distal end portion of the endoscope related to the present embodiment;

FIG. 5 is a sectional view illustrating the configuration of an optical lenses group of a lenses unit related to a first embodiment;

FIG. 6 is a view illustrating lens data of the lenses unit of the first embodiment;

FIG. 7 is a sectional view illustrating the configuration of an optical lenses group of a lenses unit related to a second embodiment; and

FIG. 8 is a view illustrating lens data of the lenses unit of the second embodiment.

### DETAILED DESCRIPTION OF THE PREFERRED EMBODIMENTS

Hereinafter, embodiments (hereinafter referred to as “present embodiment”) of a lenses unit for an endoscope and an endoscope, related to the invention, will be described in detail with reference to the accompanying drawings. The present embodiment illustrates a configuration example applied to a medical endoscope.

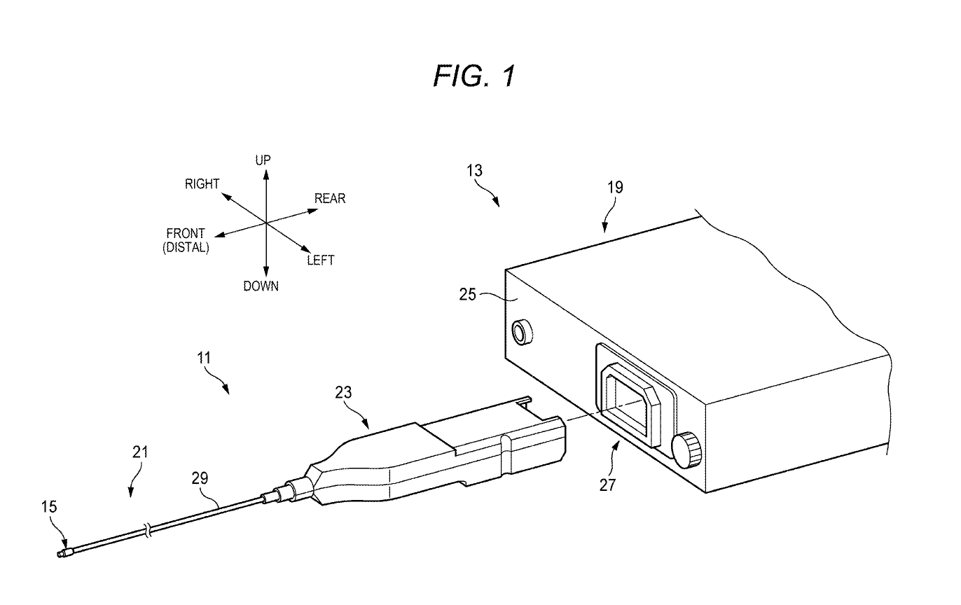

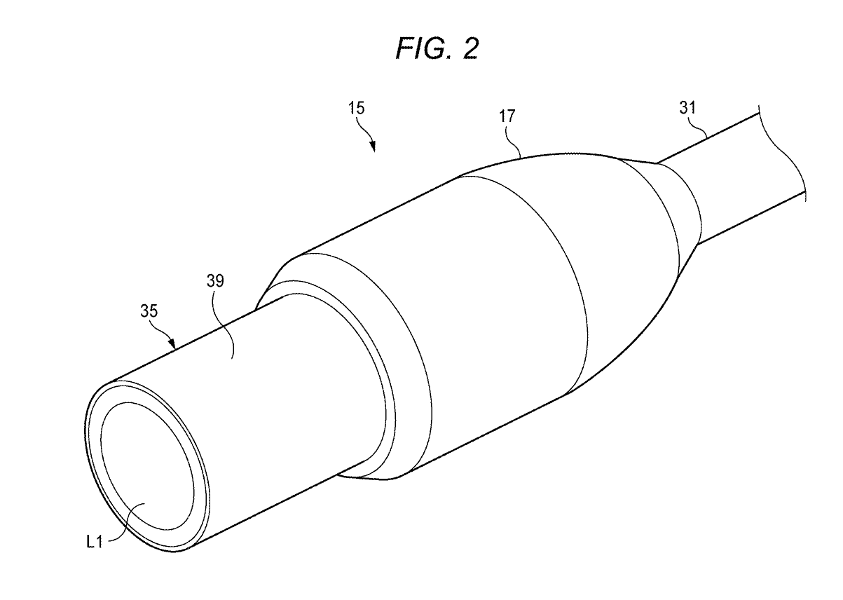

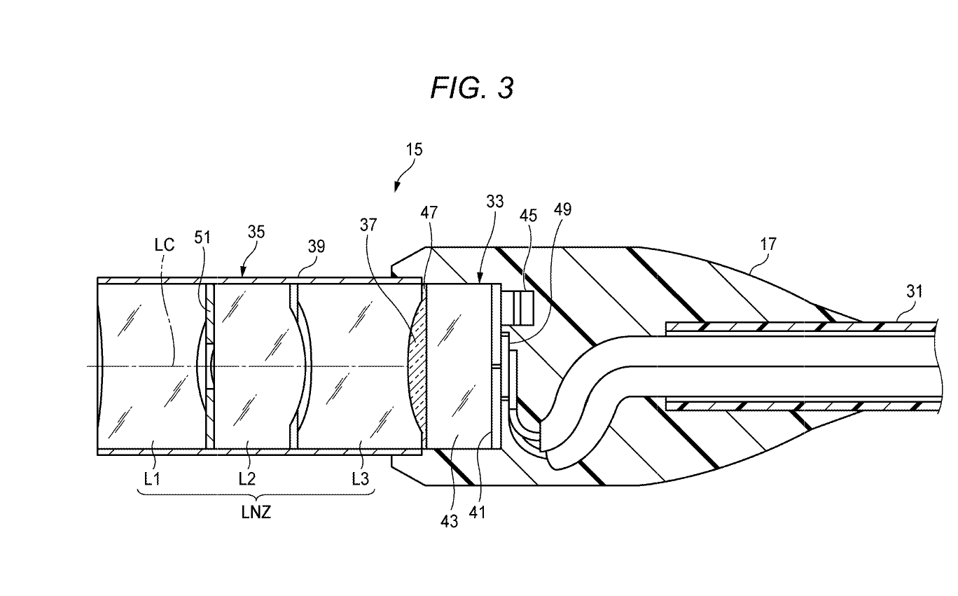

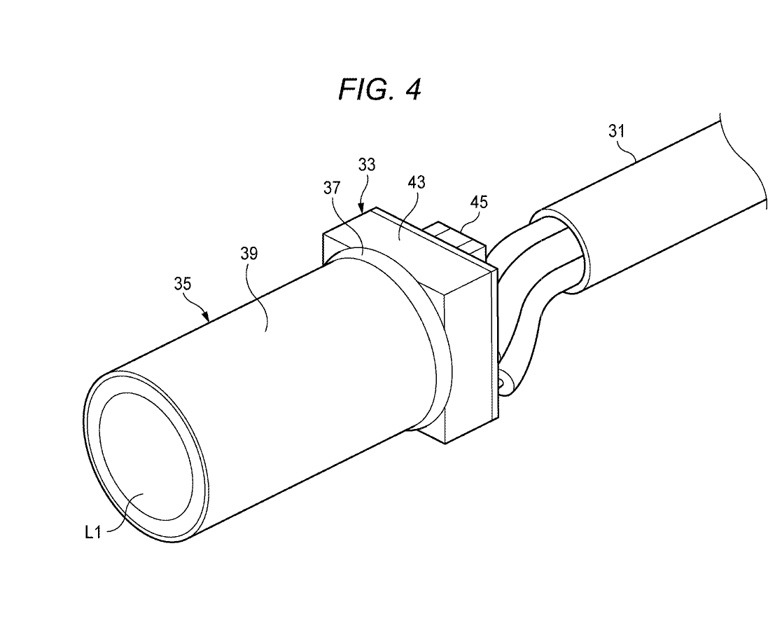

FIG. 1 is an overall configuration view of an endoscope system using the endoscope related to the embodiment of the invention. FIG. 2 is a perspective view illustrating the configuration of a distal end portion of the endoscope related to the present embodiment. FIG. 3 is a sectional view of the distal end portion of the endoscope related to the present embodiment. FIG. 4 is a perspective view illustrating the configuration of portions excluding mold resin in the distal end portion of the endoscope related to the present embodiment.

In FIG. 1, an overall configuration of endoscope system 13 including endoscope 11 and video processor 19 is illustrated in a perspective view. In FIG. 2, the configuration of distal end portion 15 of endoscope 11 illustrated in FIG. 1 is illustrated in a perspective view. In FIG. 3, the configuration of distal end portion 15 illustrated in FIG. 2 is illustrated in a sectional view. In FIG. 4, configuration excluding mold resin 17 in distal end portion 15 illustrated in FIG. 2 is illustrated in a perspective view.

Directions used for explanation in this specification follow the description of directions in the respective drawings. Here, “top” and “bottom” respectively correspond to the top and bottom of video processor 19 put on the horizontal plane, and “front (distal end)” and “rear” respectively correspond to a distal end side of insert section 21 and a base end side of plug section 23 in an endoscope body (hereinafter referred to as “endoscope 11”).

As illustrated in FIG. 1, endoscope system 13 is configured to have endoscope 11 that is a medical flexible scope, and video processor 19 that performs well-known image processing or the like on a still image and a moving image obtained by imaging the inside of an object to be observed (here, a human body). Endoscope 11 includes insert section 21 that extends in a substantially forward-rearward direction and is inserted into the object to be observed, and plug section 23 to which a rear portion of insert section 21 is connected.

Insert section 21 has flexible soft portion 29 having a rear end connected to plug section 23, and distal end portion 15 that stretches to the distal end of soft portion 29. Soft portion 29 has a suitable length corresponding to methods, such as various kinds of endoscopy and endoscopic surgery.

Video processor 19 has socket section 27 that opens to front panel 25, and includes an image processing unit and a power source unit inside the device. A rear end of plug section 23 of endoscope 11 is inserted into and engaged with socket section 27, and thereby, endoscope 11 is able to transmit and receive electric power and various signals (video signals, control signals, and the like) between endoscope 11 and video processor 19.

The above-described electric power and various signals are led to soft portion 29 and distal end portion 15 from plug section 23 via transmission cable 31 (refer to FIGS. 2 and 3) inserted into the inside of soft portion 29. Image signals output from image pickup device 33 provided at distal end portion 15 of endoscope 11 is transmitted to video processor 19 from plug section 23 via transmission cable 31. Video processor 19 performs image processing, such as color correction and gradation correction, on the image signals received in the image processing unit, and outputs the image signals subjected to the image processing to a display unit (not shown). The display unit is, for example, a monitoring device that has a display device, such as a liquid-crystal display panel, and displays an image of a photographic subject captured by endoscope 11.

As illustrated in FIGS. 2 and 3, endoscope 11 related to the present embodiment has an imaging unit mounted on distal end portion 15, and has lenses unit 35 that constitutes an imaging lens, and image pickup device 33. Lenses unit 35 is configured to house a plurality of lenses (for example, first lens L1 to third lens L3 to be described below) inside lens barrel 39. An end of lenses unit 35 on image pickup device 33 side (rear side), an outer peripheral portion of image pickup device 33, and an end of transmission cable 31 on image pickup device 33 (front side) are coated with mold resin 17 that is a sealing resin member. That is, in distal end portion 15 of insert section 21 of endoscope 11, entire image pickup device 33 and at least a portion of lenses unit 35 on image pickup device 33 side is covered with mold resin 17.

As illustrated in FIG. 3, imaging surface 41 of image pickup device 33 is covered with cover glass 43. An end of lenses unit 35 on an imaging side is adhered and fixed to cover glass 43 of image pickup device 33 with adhesive resin 37 that forms an adhesive layer. Adhesive resin 37 is constituted of, for example, transparent UV thermosetting resin, and fixes lenses unit 35 and cover glass 43 of image pickup device 33 with separating portion 47. In this case, after lenses unit 35 and image pickup device 33 are aligned with each other such that the optical axis of the lenses is made to coincide with the center of imaging surface 41, focal position adjustment (focusing) of lenses unit 35 in an optical axis direction is performed, and adhesion and fixation is performed with adhesive resin 37. This provides a configuration in which lenses unit 35 and image pickup device 33 are directly adhered and fixed to each other with adhesive resin 37. Adhesive resin 37 is, for example, an adhesive of a type in which heat treatment is required in order to obtain final hardness, but curing proceeds to a certain degree of hardness even by ultraviolet irradiation.

Mold resin 17 is made of, for example, a resin material having a light blocking effect, such as black resin. In this way, the connected and fixed region between lenses unit 35 and image pickup device 33 has a double structure in which an outer peripheral portion of adhesive resin 37 having a light-transmitting property, such as a transparent material that transmits rays of a subject image, is provided with and coated with mold resin 17 having a light blocking effect, such as black.

Circuit board 49 is mounted on the surface of image pickup device 33 opposite (rear side) to cover glass 43, and has capacitor 45 for a countermeasure against static electricity attached thereto. Transmission cable 31 is electrically connected to a rear portion of circuit board 49, and the connection region of circuit board 49 is covered with mold resin 17 for sealing. In the subsequent description, the term “adhesive” is used not in a strict meaning indicating a substance used to adhere surfaces of solid bodies to each other but in a broad meaning indicating a substance that can be used for connection of two objects, or a substance having a function as a sealing agent when the cured adhesive has high barrier properties against gas and liquid.

Lens barrel 39 is made of a cylinder material having high rigidity, for example, a metallic tubular member. By using a hard material for lens barrel 39, distal end portion 15 constitutes a hard part. As a metallic material that constitutes lens barrel 39, for example, nickel is used. Nickel has a relatively high modulus of rigidity and high corrosion resistance, and is suitable as a material that constitutes distal end portion 15. Instead of nickel, for example, a copper nickel alloy may be used. The copper nickel alloy also has high corrosion resistance, and is suitable as a material that constitutes distal end portion 15. As a metallic material that constitutes lens barrel 39, a material that can be manufactured by electroforming (electroplating) is preferably selected. Here, the reason why the electrocasting is used is because the dimensional precision of a member that is manufactured by electrocasting is as extremely high, at less than (so-called submicron precision) 1 μm, and the variation when a number of members are manufactured is small. As will be described below, lens barrel 39 is an extremely small member, and a dimensional error between the external and internal diameters thereof influences the optical performance (image quality) of endoscope 11. By constituting lens barrel 39 of, for example, a nickel electroforming pipe, endoscope 11 capable of securing high dimensional precision in spite of a small diameter and capturing a high-definition image is obtained.

The plurality of (three in an illustrated example) lenses (first lens L1 to third lens L3) formed of an optical material (glass, resin, or the like), and aperture 51 sandwiched between first lens L1 and second lens L2 are assembled into lens barrel 39 in a state where they are brought into close contact with each other in the direction of optical axis LC. First lens L1 and third lens L3 are fixed to the inner peripheral surface of lens barrel 39 with an adhesive over their whole circumferences. Since a front end of lens barrel 39 is hermetically closed (sealed) by first lens L1, and a rear end is hermetically closed (sealed) by third lens L3, lens barrel 39 is configured such that air or moisture does not enter the inside of the lens barrel. Therefore, air or the like cannot escape from one end of lens barrel 39 to the other end thereof. In the subsequent description, first lens L1 to third lens L3 are altogether referred to as optical lenses group LNZ. Optical lenses group LNZ formed by the plurality of lenses is not limited to the three-lens configuration, and the number of lenses is arbitrary so long as configurations having a front group lens and a rear group lens, such as two or more lenses or four or more lenses, are provided.

As illustrated in FIGS. 3 and 4, image pickup device 33 is constituted of, for example, an imaging device, such as a small-sized Charge Coupled Device (CCD) or a Complementary Metal-Oxide Semiconductor (CMOS), forms a square shape as seen from the forward-rearward direction. In this case, imaging surface 41 that forms a square shape in a front view is provided at a central portion of image pickup device 33. The light that has entered the imaging unit from the outside forms an image on imaging surface 41 of image pickup device 33 by optical lenses group LNZ within the lens barrel. Circuit board 49 mounted on a rear portion (back side) of image pickup device 33 has an outer shape slightly smaller than image pickup device 33 as seen from the rear. Image pickup device 33 includes, for example, a Land Grid Array (LGA) on a back surface thereof, and is electrically connected to an electrode pattern formed on circuit board 49.

Here, a circle that forms an outer periphery of lens barrel 39 has a relationship in which the circle is substantially inscribed on the square formed by image pickup device 33 and is circumscribed on the square formed by imaging surface 41. The positions of the center (an intersection point between diagonal lines of imaging surface 41) of imaging surface 41, the center (the center of the circle formed by the inner periphery of lenses unit 35) of lenses unit 35, and the center (the center of the circle formed by the outer periphery of lens barrel 39) of lens barrel 39 coincide with each other, and the optical axis LC passes through these positions. More exactly, a normal line passing through the center of imaging surface 41 is optical axis LC, and lenses unit 35 is aligned with image pickup device 33 such that optical axis LC passes through the center of lenses unit 35.

Next, a configuration example of optical lenses group LNZ (first lens L1 to third lens L3) in lenses unit 35 of endoscope 11 will be described.

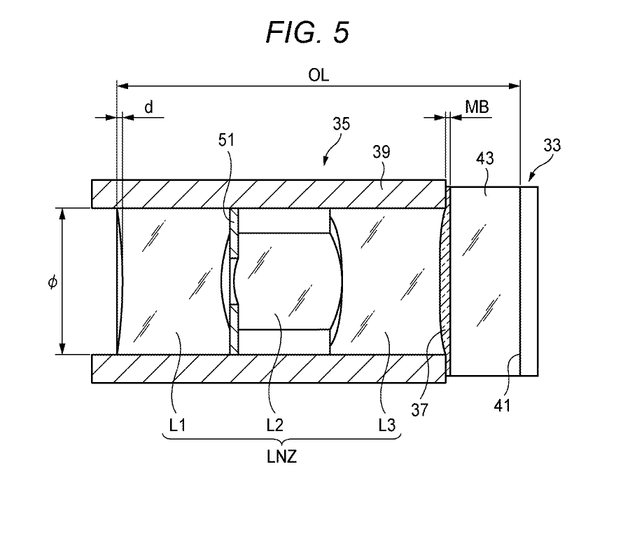

FIG. 5 is a sectional view illustrating the configuration of the optical lenses group of the lenses unit related to the first embodiment.

The first embodiment illustrates a first configuration example of optical lenses group LNZ of lenses unit 35 in distal end portion 15 of the endoscope. In lenses unit 35 of the present embodiment, first lens L1 functions as the front group lens, and second lens L2 and third lens L3 function as the rear group lens. Here, first lens L1 is a leading lens of optical lenses group LNZ, and third lens L3 is a final lens of optical lenses group LNZ. Lenses unit 35 is configured such that, in order from a photographic subject side toward the imaging side, first surface L1R1 of first lens L1 that is the forefront surface has a concave surface, second surface L1R2 has a concave surface, first surface L2R1 of second lens L2 has a convex surface, second surface L2R2 has a convex surface, first surface L3R1 of third lens L3 has a concave surface, and second surface L3R2 that is a final surface has a concave surface.

Aperture 51 is provided between first lens L1 and second lens L2, that is, between the front group lens and the rear group lens. A gap between second surface L3R2 (final surface) of third lens L3 that is the concave surface, and cover glass 43 of image pickup device 33 is filled with adhesive resin 37, which in turn forms the adhesive layer.

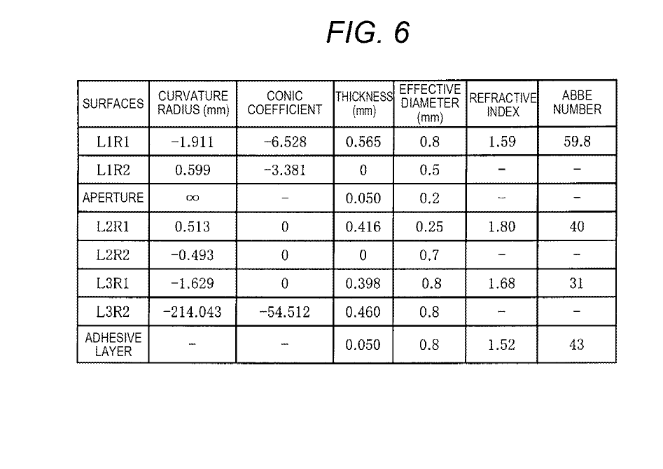

FIG. 6 is a view illustrating lens data of the lenses unit of the first embodiment. In FIG. 6, surfaces correspond to respective surfaces L1R1 to L3R2 of first lens L1 to third lens L3, aperture 51, and the adhesive layer (adhesive resin 37), respectively, and the curvature radius (mm), the conic coefficient, and the effective diameter (mm) of the respective surfaces are shown. The thickness (mm) shows the distance (thickness) from a relevant surface to the next surface in the optical axis direction in the optical center, and the refractive index and the Abbe number show the refractive index and the Abbe number of an optical member that forms the relevant surface. Here, the external diameter φ (external diameter of first lens L1, second lens L2 and third lens L3) of optical lenses group LNZ is about φ=0.9 to 1.0 mm. However, it may be about φ=0.7 to 1.2 mm. The thickness of cover glass 43 of image pickup device 33 is 0.4 mm.

In the first embodiment, focal distance fₑₗ of entire optical lenses group LNZ is set to fₑₗ=0.58 mm, focal distance f_{F} of the front group lens (first lens L1) is set to f_{F}=−0.714, and focal distance f_{B} of the rear group lens (second lens L2 and third lens L3) is set to f_{B}=0.481. If total optical length OL of optical lenses group LNZ is defined as the length from the forefront surface (first surface L1R1 of first lens L1) of the leading lens to the imaging surface (rear end surface of cover glass 43 of image pickup device 33 on the imaging side), entire optical length OL=2.287 mm is established, however, entire optical length OL may also be 2.0 mm to 2.7 mm.

If the length from a peripheral portion end surface of the final surface (second surface L3R2 of third lens L3) of the final lens to a front end surface of cover glass 43 of image pickup device 33 on the photographic subject side is defined as metal back MB, metal back MB=0.04 mm is established. However, MB may be 0.01 mm to 0.1 mm. Metal back MB may also be referred to as a back focus depending on the irregularities of the final surface of the final lens. Here, back focus BF and metal back MB will be unified and described as metal back MB, using metal back MB as a parameter of the concept including back focus BF. As illustrated in FIG. 6, since the thickness at the optical center of the adhesive layer is 0.05 mm, however, the adhesive layer may be 0.01 mm to 0.1 mm, and second surface L3R2 of third lens L3 is the concave surface, metal back MB equivalent to the distance from the peripheral portion end surface of second surface L3R2 to the front end surface of cover glass 43 becomes shorter than the optical center.

In this case, fₑₗ/f_{F}=−0.812, LAB=1.206, and OL/MB=38.12 are established, and

the relationship of fₑₗ/f_{F}<0, fₑₗ/f_{B}>0, and OL/MB>7.0 is satisfied.

Curvature radius rLbR2 (rL3R2) of the imaging-side final surface (second surface L3R2 of third lens L3) of the rear group lens is rLbR2=−214.043≠∞. Refractive index n_{be} (n3) of the final lens (third lens L3) of the rear group lens is n_{be}=1.68, refractive index n_{ad} of the adhesive layer is n_{ad}=1.52, and n_{be}≠n_{ad} is established.

Since Abbe number v_{be} (v3) of the final lens (third lens L3) of the rear group lens is v_{be}=31>25, and the refractive index of the final lens is n_{be}=1.68, the relationship of 1.40<n_{be}<1.90 is satisfied.

The forefront surface (first surface L1R1 of first lens L1) of the front group lens is a concave surface, and sag (SAG) amount d of the concave surface is d=0.021 mm, however, it may be d=−0.12 mm to 0.12 mm. Here, sag amount d of the concave surface and lens external diameter φ of optical lenses group LNZ becomes d/φ=0.021 when φ=1.0 mm is established, and the relationship of −0.1<d/φ<0.1 is satisfied.

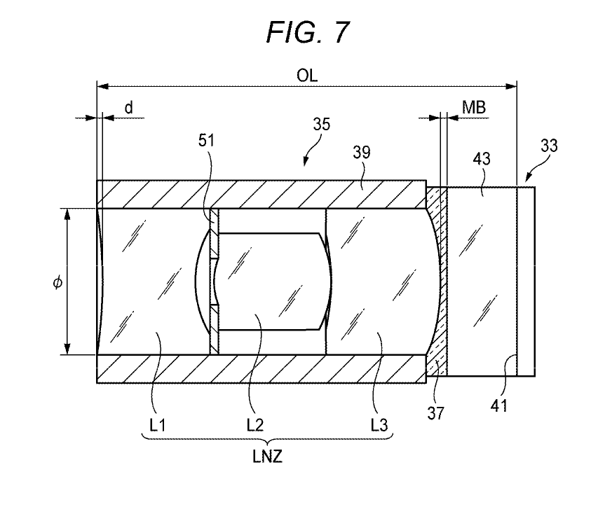

FIG. 7 is a sectional view illustrating the configuration of an optical lenses group of a lenses unit related to a second embodiment.

The second embodiment illustrates a second configuration example of optical lenses group LNZ of lenses unit 35 in distal end portion 15 of the endoscope. In lenses unit 35 of the present embodiment, similar to the first embodiment, first lens L1 functions as the front group lens, and second lens L2 and third lens L3 function as the rear group lens. Here, first lens L1 is a leading lens of optical lenses group LNZ, and third lens L3 is a final lens of optical lenses group LNZ. Lenses unit 35 is configured such that, in order from a photographic subject side toward the imaging side, first surface L1R1 of first lens L1 that is the forefront surface has a concave surface, second surface L1R2 has a concave surface, first surface L2R1 of second lens L2 has a convex surface, second surface L2R2 has a convex surface, first surface L3R1 of third lens L3 has a concave surface, and second surface L3R2 that is a final surface has a convex surface.

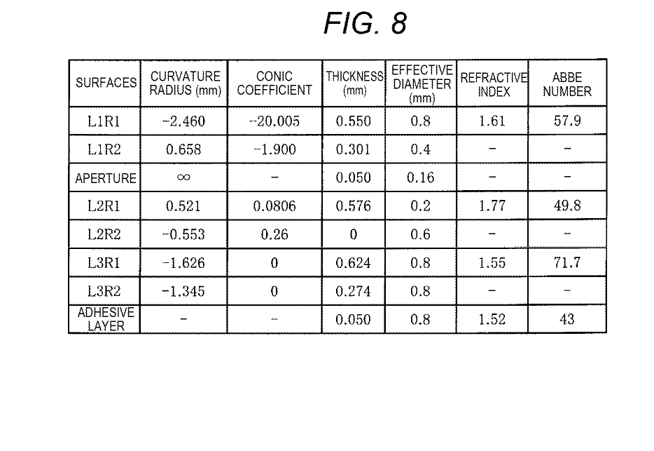

FIG. 8 is a view illustrating lens data of the lenses unit of the second embodiment. In FIG. 8, similar to FIG. 6, respective surfaces L1R1 to L3R2, the aperture, the curvature radius (mm), the unique coefficient, the thickness (mm), the effective diameter (mm) in the adhesive layer, and the refractive index and the Abbe number of an optical member that form a relevant surface are illustrated. Here, the external diameter φ (external diameter of first lens L1 and third lens L3) of optical lenses group LNZ is about φ=0.9 to 1.0 mm, however, it may also be φ=0.7 to 1.2 mm. The thickness of cover glass 43 of image pickup device 33 is 0.4 mm, but the thickness may be 0.2 mm to 0.8 mm.

In the second embodiment, focal distance fₑₗ of entire optical lenses group LNZ is set to fₑₗ=0.61 mm, focal distance f_{F} of the front group lens (first lens L1) is set to f_{F}=−0.79, and focal distance f_{B} of the rear group lens (second lens L2 and third lens L3) is set to f_{B}=0.501. Total optical length OL of optical lenses group LNZ is OL=2.300 mm. Metal back MB is MB=0.08 mm, however, it may be MB=0.01 mm to 0.1 mm. As illustrated in FIG. 8, since the thickness at the optical center of the adhesive layer is 0.05 mm, and second surface L3R2 of third lens L3 is the convex surface, metal back MB equivalent to the distance from the peripheral portion end surface of second surface L3R2 to the front end surface of cover glass 43 becomes longer than the optical center.

In this case, fₑₗ/f_{F}=−0.772, fₑₗ/f_{B}=1.217, and OL/MB=37.1 are established, and

the relationship of fₑₗ/f_{F}<0, fel/f_{B}>0, and OL/MB>7.0 is satisfied.

Curvature radius rLbR2 (rL3R2) of the imaging-side final surface (second surface L3R2 of third lens L3) of the rear group lens is rLbR2=−1.345≠∞. Refractive index n_{be} (n3) of the final lens (third lens L3) of the rear group lens is n_{be}=1.55, refractive index n_{ad} of the adhesive layer is n_{ad}=1.52, and n_{be}≠n_{ad} is established.

Since Abbe number v_{be} (v3) of the final lens (third lens L3) of the rear group lens is v_{be}=71.7>25, and the refractive index of the final lens is n_{be}=1.55, the relationship of 1.40<n_{be}<1.90 is satisfied.

The forefront surface (first surface L1R1 of first lens L1) of the front group lens is a concave surface, and sag amount d of the concave surface is d=0.030 mm. Here, sag amount d of the concave surface and lens external diameter φ of optical lenses group LNZ become d/φ=0.030 when φ=1.0 mm is established, and the relationship of −0.1<d/φ<0.1 is satisfied.

Here, an example of the dimensions of the lenses unit for an endoscope and the endoscope related to the present embodiment is illustrated. Numerical values illustrated below show one specific example, and various examples can be considered according to applications, usage environments, or the like. As an example, in lenses unit 35, as in the above example, total optical length OL is 2.2 mm to 2.3 mm, lens external diameter φ is 1.0 mm, the longitudinal dimension of the imaging unit including lens barrel 39 and image pickup device 33 is about 2.5 mm, and the external diameter is about 1.1 mm. The length of distal end portion 15 on which the imaging unit is loaded is about 3.5 mm, and the maximum external diameter thereof is about 1.5 mm.

The above-described present embodiment is configured such that lenses unit 35 for an endoscope, the front group lens (first lens L1) having negative power, and the rear group lens (second lens L2, third lens L3) having positive power are provided, and total optical length OL is larger than metal back MB. Accordingly, metal back MB becomes smaller than total optical length OL, and the distance from the final surface of optical lenses group LNZ to the front end surface of cover glass 43 of image pickup device 33 becomes short. Therefore, it is possible to adopt a configuration in which optical lenses group LNZ and cover glass 43 of image pickup device 33 are directly adhered and fixed to each other with adhesive resin 37. Hence, the imaging unit can be made into a structure with high strength and with a small number of parts, the imaging lens with a short focal distance can be realized, and shortening of the length of the imaging lens and downsizing can be achieved.

In this way, in the present embodiment, a further small-sized imaging unit is realized by reducing the diameter of the imaging lens, achieving an optical design in which the length of the imaging unit including the imaging lens and the image pickup device is shortened, and adopting a structure in which the imaging lens and the image pickup device are directly fixed with the adhesive layer.

In the structure in which optical lenses group LNZ and cover glass 43 are directly fixed with the adhesive layer of adhesive resin 37, the final surface (L3R2) of optical lenses group LNZ is formed into a curved surface, and the refractive indexes of the final lens (third lens L3) and the adhesive layer are made different from each other. Accordingly, since the final surface of optical lenses group LNZ can be made to have refractive power, the convergence of the rays from a photographic subject that passes through lenses unit 35 can be further enhanced, this can contribute to a reduction in aberration. The number of lenses for obtaining required optical performance regarding the optical performance (resolution, chromatic aberration, distortion, or the like) of optical lenses group LNZ can be reduced. Therefore, it is possible to obtain desired optical performance while achieving downsizing and cost reduction of the imaging lens.

By making Abbe number v_{be} (v3) of the final lens (third lens L3) larger than 25 and making refractive index n_{be} (n3) fall within a range of 1.40 to 1.90, the chromatic aberration of magnification can be reduced, and can be made smaller than the pixel pitch of image pickup device 33. Therefore, color blurring in a peripheral portion of a captured image can be reduced.

By making the forefront surface (L1R1) of optical lenses group LNZ into the concave surface and setting sag amount d of the concave surface such that the absolute value of relative ratio d/φ to lens external diameter φ becomes smaller than 0.1, the forefront surface can be brought close to a plane and adhesion of dirt in using the endoscope can be reduced. The forefront surface (L1R1) of optical lenses group LNZ may be a convex surface. In this case, the sag amount of the convex surface is set such that the absolute value of d/φ becomes smaller than 0.1.

Although the various embodiments have been described above with reference to the drawings, the invention is not limited to these examples. It is apparent that a person skilled in the art may find various alternations and modifications within the scope of the appended claims, and it should be understood that they will naturally come under the technical scope of the present invention. The respective constituent elements in the above embodiments may be arbitrarily combined without departing from the scope of the invention.

What is claimed is:

1. A lenses unit used for an endoscope, comprising:
a lens barrel;
a front group lens and a rear group lens housed inside the lens barrel; and
an aperture arranged between the front group lens and the rear group lens,
wherein an imaging-side final surface of the rear group lens has a structure in which the imaging-side final surface is fixed to a cover glass of an image pickup device with an adhesive layer, and
wherein a focal distance f_{F} of the front group lens, a focal distance f_{B} of the rear group lens, a focal distance fₑₗ of an entire optical lenses group including the front group lens and the rear group lens, a total optical length OL of the optical lenses group, and a metal back MB of the optical lenses group satisfy a relationship of
fₑₗ/f_{F}<0, fₑₗ/f_{B}>0, and OL/MB>7.0.

2. The lenses unit of claim 1,
wherein a curvature radius rLbR2 of the imaging-side final surface of the rear group lens is rLbR2≠∞, and
wherein a refractive index n_{be} of a final lens of the rear group lens on an imaging side and a refractive index n_{ad} of the adhesive layer when the rear group lens is fixed by the adhesive layer are n_{be}≠n_{ad}.

3. The lenses unit of claim 1,
wherein an Abbe number v_{be} of a final lens of the rear group lens on an imaging side is v_{be}>25, and
wherein a refractive index n_{be} of a final lens of the rear group lens on the imaging side is 1.40<n_{be}<1.90.

4. The lenses unit of claim 1,
wherein the forefront surface of the front group lens on a photographic subject side is a concave surface or a convex surface, and a sag amount d of the concave surface or the convex surface and a lens external diameter φ of the optical lenses group satisfy a relationship of −0.1<d/φ<0.1.

5. An endoscope comprising:
a lenses unit;
an image pickup device in which an imaging surface is covered with a cover glass;
an imaging-side final surface of the rear group lens in the lenses unit for an endoscope; and
an adhesive layer formed by an adhesive resin with which the cover glass of the image pickup device is fixed,
wherein the lenses unit includes:
a lens barrel;
a front group lens and a rear group lens housed inside the lens barrel; and
an aperture arranged between the front group lens and the rear group lens,
wherein an imaging-side final surface of the rear group lens has a structure in which the imaging-side final surface is fixed to the cover glass of the image pickup device with the adhesive layer, and
wherein a focal distance f_{F} of the front group lens, a focal distance f_{B} of the rear group lens, a focal distance fₑₗ of an entire optical lenses group including the front group lens and the rear group lens, a total optical length OL of the optical lenses group, and a metal back MB of the optical lenses group satisfy a relationship of
fₑₗ/f_{F}<0, fₑₗ/f_{B}>0, and OL/MB>7.0.

6. The endoscope of claim 5,
wherein a curvature radius rLbR2 of the imaging-side final surface of the rear group lens is rLbR2≠∞, and
wherein a refractive index n_{be} of a final lens of the rear group lens on an imaging side and a refractive index n_{ad} of the adhesive layer when the rear group lens is fixed by the adhesive layer are n_{be}≠n_{ad}.

7. The endoscope of claim 5,
wherein an Abbe number v_{be} of a final lens of the rear group lens on an imaging side is v_{be}>25, and
wherein a refractive index n_{be} of a final lens of the rear group lens on the imaging side is 1.40<n_{be}<1.90.

8. The endoscope of claim 5,
wherein the forefront surface of the front group lens on a photographic subject side is a concave surface or a convex surface, and a sag amount d of the concave surface or the convex surface and a lens external diameter φ of the optical lenses group satisfy a relationship of −0.1<d/φ<0.1.

9. An endoscope comprising:
a lens unit including a lens barrel, a front group lens and a rear group lens housed inside the lens barrel, and an aperture arranged between the front group lens and the rear group lens;
an imaging element in which an imaging surface is covered with cover glass;
an adhesive layer formed by an adhesive resin with which an imaging-side final surface of the rear group lens in the lens unit and the cover glass of the imaging element are fixed,
wherein the imaging-side final surface of the rear group lens and an imaging-side end surface of the lens barrel are structured so as to be fixed to the cover glass via the adhesive layer, and
wherein a focal distance f_{F} of the front group lens, a focal distance f_{B} of the rear group lens, a focal distance fₑₗ of an entire optical system including the front group lens, the rear group lens, the adhesive layer, and the cover glass, a total optical length OL corresponding to a distance from a forefront surface of the front group lens on a photographic subject side to an imaging-side rear end surface of the cover glass, and a metal back MB corresponding to a distance from the imaging-side final surface of the rear group lens to a front end surface of the cover glass on the photographic subject side satisfy a relationship of
fₑₗ/f_{F}<0, fₑₗ/f_{B}>0, and OL/MB>7.0.

10. The endoscope of claim 9,
wherein at least an imaging-side end portion of the lens barrel, the adhesive layer, and the cover glass are covered with mold resin.
